(19) United States
(12) Patent Application Publication
(10) Pub. No.: US 2008/0166384 A1
(43) Pub. Date: Jul. 10, 2008

(54) STETHOSCOPE HEAD COVER AND ASSOCIATED METHOD

(76) Inventor: Darren Jones, American Fork, UT (US)

Correspondence Address: THORPE NORTH & WESTERN, LLP., P.O. Box 1219, SANDY, UT, 84091-1219, US

(21) Appl. No.: 11/968,805

(22) Filed: Jan. 3, 2008

Related U.S. Application Data
(60) Provisional application No. 60/878,780, filed on Jan. 5, 2007.

(51) Int. Cl. A61K 33/34; A61K 33/24; A61K 31/045; A61K 33/38

(52) U.S. Cl. 424/402; 424/600; 514/724; 428/131; 424/618; 424/649; 424/638

(57) ABSTRACT

A stethoscope head cover configured to envelop a head of a stethoscope includes a cover member with a cavity therein. The cover member includes first and second openings. The first opening has a size sufficient to slidably engage a cord associated with the head of the stethoscope, and the second opening has a size sufficient to allow the head of the stethoscope to be extended therethrough. A stethoscope head cover with a cavity therein and a first opening having a size sufficient to allow the head of the stethoscope to be extended therethrough. The head cover can further include a biocidal environment within the cavity of the cover member. A method of sanitizing a stethoscope head includes providing a stethoscope head cover having a biocidal environment within a cavity of the stethoscope, and engaging a stethoscope head with the stethoscope head cover.

### PRIORITY CLAIM

This application claims priority to U.S. Patent Application Ser. No. 60/878,780, filed on Jan. 5, 2007, which is incorporated herein by reference.

### FIELD OF THE INVENTION

The present invention relates generally to covers for medical instruments and more specifically to stethoscope head covers and associated methods for sanitizing the head of a stethoscope. Accordingly, the present invention involves the material and chemical science fields.

### BACKGROUND OF THE INVENTION

Medical instruments such as stethoscopes are commonly used in the medical and health care industry. Stethoscopes are used by doctors and other health care professionals to listen to sounds within a patient's body such as a heartbeat and breathing sounds in the lungs. This is accomplished by placing the stethoscope directly on the patient's skin. Unlike some medical instruments, a single stethoscope is not disposable, but is used on multiple patients, most often without cleaning the stethoscope between uses. For this reason, harmful and disease causing microorganisms such as bacteria, viruses and fungi can be inadvertently transferred from one patient to another patient. In addition to being a source for transfer of harmful microorganisms, stethoscopes are also known for being cold when coming into contact with a patient's skin. Accordingly, many patients complain that it is uncomfortable to have a stethoscope head placed against his or her skin.

### SUMMARY OF THE INVENTION

The present invention provides a stethoscope head cover configured to envelop a head of a stethoscope. The stethoscope head cover includes a cover member with a cavity therein. The cover member can include one or more openings to provide access to the cavity. According to one aspect, the cover member may have a first opening with a size sufficient to slidably engage a cord associated with the head of the stethoscope, and a second opening having a size sufficient to allow the head of the stethoscope to be extended therethrough. In this embodiment, the cover may be disengaged from the stethoscope head by extending the head through the second opening and sliding the cover up the cord through the first opening. After use, the cover can be reengaged with the stethoscope head by simply sliding the cover back down the cord using the first opening and retracting the head back through the second opening and into the cover.

Various arrangements may be made regarding the first and second openings in this embodiment of the invention. In one aspect, the openings may be substantially equal in size. In another aspect, the first opening may be sufficiently small so as to prevent the stethoscope head from passing therethrough, while the second opening is sufficiently large to allow the head to extend or pass therethrough. In further aspects, the size of one or both openings may be adjustable by a number of mechanisms, including elastics, drawstrings, hook and loop fasteners, etc.

In yet another embodiment, the cover may have a closable slit that extends either part way, or entirely between and connects the two openings. In such case, the cover can be partially or fully opened to disengage it completely from the stethoscope, for cleaning, treatment, disposal, etc., by unfastening and opening the slit.

The stethoscope head cover can comprise a soft and flexible material. In one embodiment, the soft and flexible material can include a polymeric material. Additionally, the soft and flexible material can include a fabric material. Alternatively, the cover member can comprise a rigid material. The rigid material can include a polymeric material, such as plastic. In a preferred aspect, the cover member can comprise an elastic material, thus enabling the cover member to reversibly deform.

The stethoscope head cover of the present invention can also comprise a biocidal environment within the cavity of the cover member. The biocidal environment can be provided by a number of mechanisms, including the nature of the cover member material itself, an antimicrobial lining contiguous with an inner surface of the cover member, or a number of other mechanisms.

In one aspect, the antimicrobial lining and/or the material comprising the cover member can include, or be configured to hold a biocidal agent. The biocidal agent can comprise at least one member selected from the group consisting of alcohols, quaternary ammonium salts, metal ions, chlorine, bromine and biocidal polymers and combinations and mixtures thereof. In a preferred aspect, the biocidal agent can be chlorine, bromine, or combinations and mixtures thereof. In another preferred aspect, the biocidal agent can be a metal, such as copper or silver. Thus, the stethoscope head can be sanitized by upon insertion into the cover.

Various methods of making and using a stethoscope head cover are also encompassed by the present invention. In one aspect, a method of sanitizing a stethoscope head can include providing a stethoscope head cover in accordance with any of the embodiments set forth above, and engaging a stethoscope head with the stethoscope head cover.

There has thus been outlined, rather broadly, various features of the invention so that the detailed description thereof that follows may be better understood, and so that the present contribution to the art may be better appreciated. Other features of the present invention will become clearer from the following detailed description of the invention, taken with the accompanying claims, or may be learned by the practice of the invention.

### BRIEF DESCRIPTION OF THE DRAWINGS

FIG. 1 is a front sectional view of a stethoscope head cover, shown on a stethoscope;

FIG. 2 is a front perspective view of a stethoscope head cover, wherein the cover has a first opening and a second opening;

FIG. 3 is a front view of a stethoscope head cover, wherein the cover has only one opening;

### DETAILED DESCRIPTION OF THE INVENTION

Before the present stethoscope head cover and accompanying methods of use and manufacture are disclosed and described, it is to be understood that this invention is not limited to the particular process steps and materials disclosed herein, but is extended to equivalents thereof as would be recognized by those ordinarily skilled in the relevant arts. It should also be understood that terminology employed herein is used for the purpose of describing particular embodiments only and is not intended to be limiting.

It must be noted that, as used in this specification and the appended claims, the singular forms “a,” “an,” and, “the” include plural referents unless the context clearly dictates otherwise. Thus, for example, reference to “the material” or “a cloth” includes reference to one or more of such materials.

### DEFINITIONS

In describing and claiming the present invention, the following terminology will be used in accordance with the definitions set forth below.

As used herein, “cover member” refers to a cover or container of a material that is capable of having a stethoscope enveloped therein. In one aspect, the cover may be of a flexible material that is durable and waterproof so as to protect a stethoscope head within the cover member.

As used herein, “fastener” refers to a structure or method which is capable of at least partially closing, and holding closed, an opening of a cover member, or securing a slit extending partially or fully therebetween. In some aspects, the opening may be entirely closed and in some aspects, made water tight. Examples of specific fasteners include without limitation, snaps, hooks, straps, cords, hook and loop fasteners (i.e. Velcro®), buttons, drawstrings, buckles, clasps, and zippers, among others.

As used herein, “plastic” refers to a rigid or semi-rigid polymeric material that can be extruded, molded, or cast into a number of different shapes. A wide variety of plastics are known to those of ordinary skill in the art, a number of which may be selected in order to provide a head cover with specifically desired characteristics.

As used herein, “metal” refers to a chemical element or substance (such as gold, silver, or copper) which is crystalline when solid. A wide variety of metals are known to those of ordinary skill in the art and may be selected for use in connection with the present invention based on a variety of characteristics, including antimicrobial properties. As referenced herein, “metal” can refer to a single metal, ions of metal, metal alloys, and mixtures and composites of metals.

As used herein, the terms “biocidal” and “antimicrobial” are interchangeable and refer to any composition that kills, destroys, inhibits growth, or decreases the activity of microorganisms, including bacteria, viruses, and fungi.

As used herein, the term “sanitize” refers to making an object more sanitary by killing, destroying, inhibiting growth, or decreasing the activity of microorganisms, including bacteria, viruses, and fungi that may be present on or around the object. The term sanitize also refers to making an object more sanitary by cleaning, disinfecting or sterilizing the object. An object that has been sanitized may or may not also be sterile.

Sizes, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited.

As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. This same principle applies to ranges reciting only one numerical value. Furthermore, such an interpretation should apply regardless of the breadth of the range or the characteristics being described.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a cavity that is “substantially free of” harmful bacteria would either completely lack harmful bacteria, or so nearly completely lack harmful bacteria that the effect would be the same as if it completely lacked harmful bacteria. In other words, an area that is “substantially free of” an ingredient or element may still contain such item as long as there is no measurable effect thereof.

As used herein, the term “about” is used to provide flexibility to a numerical range endpoint by providing that a given value may be “a little above” or “a little below” the endpoint.

### THE INVENTION

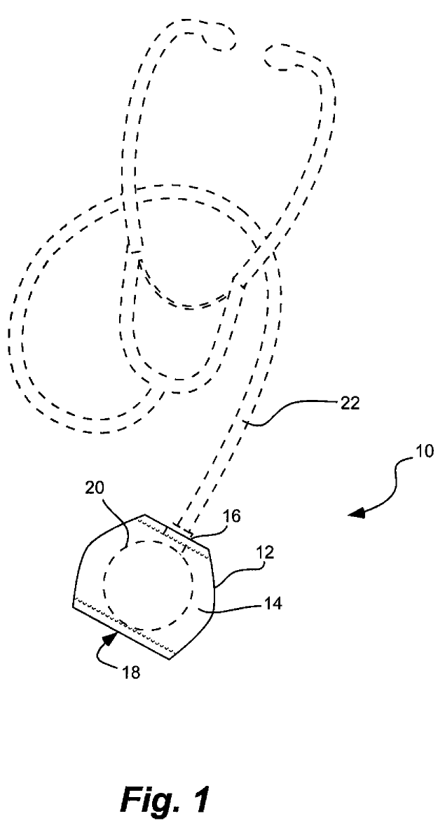

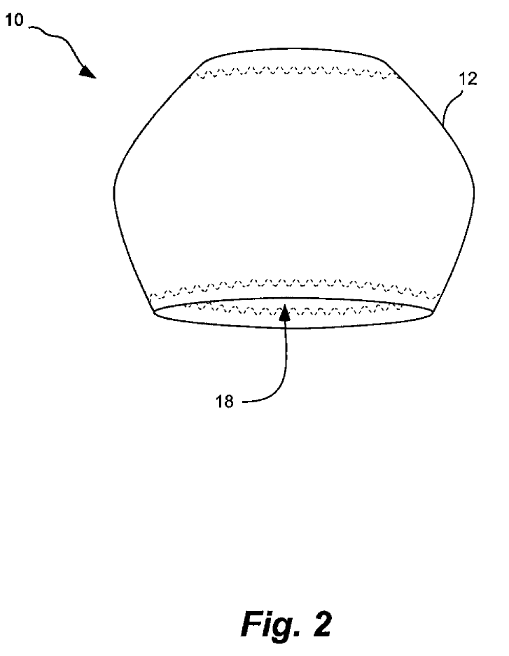

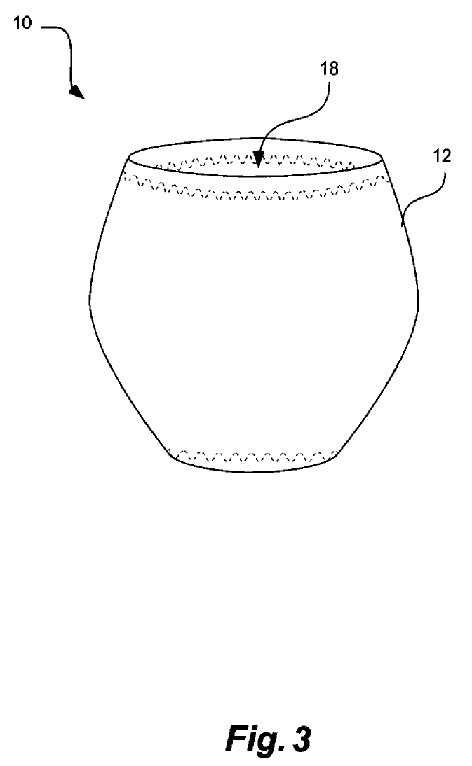

The present invention provides a stethoscope head cover 10 configured to envelop the head of a stethoscope. As shown in FIGS. 1-3, the stethoscope head cover can include a cover member 12 with a cavity 14 therein. The cover member can include one or more openings. According to one aspect, the cover member can have an opening 18 with a size sufficient to allow the head 20 of the stethoscope to be extended therethrough. The cover member can also have a second opening 16 with a size sufficient to slidably engage a cord associated with the head of the stethoscope. The opening or first opening is best viewed in FIG. 2. In an alternative aspect, the cover member may have only a single opening 18 having a size sufficient to allow the head of the stethoscope to be passed or extended therethrough. FIG. 3 shows an embodiment of a stethoscope head cover having only a single opening.

In use, the stethoscope head cover 10 discussed herein substantially envelops the stethoscope head 20 when not in use and can be removed from off the head when the stethoscope is to be used. When the embodiment having only a single opening 18 is used, as shown in FIG. 3, the single opening provides both the access into and out of the interior cavity 14 of the cover 10. Here, the opening can have a size sufficient to allow ingress and egress for the head 20 of the stethoscope. The opening can be a single slit or an arrangement of slits in the material of the cover member so long as the slit can stretch to allow passage of the stethoscope head therethrough. In addition, an attachment member, such as a cord or clip, can be coupled to the cover member so that when the cover member is removed from the head it can remain attached to the stethoscope cord, thereby avoiding contact with potentially contaminated surfaces.

When the embodiment having two openings is used, as shown in FIGS. 1-2, the opening 18 is used to provide access into and out of the interior cavity. Here the head cover need not be disengaged from the stethoscope entirely when the stethoscope is in use. Instead, the second opening 16 can be used to allow the cover 10 to slide up the stethoscope cord 22, which connects the head of the stethoscope to its earpieces. The cover may then reengage the stethoscope head 20 by simply sliding it back down the cord after use of the stethoscope is complete.

Referring to the physical and structural embodiments of the stethoscope head cover 10, the cover member 12 can be of a variety of shapes and sizes. Circular, square, rectangular, triangular, hexagonal, and other custom designed shapes and geometrical configurations may be used. By way of example, the cover can be rounded and have a curvature which corresponds to the curvature of the stethoscope head so that substantially all of the surface of the stethoscope head is in direct contact with an inside surface of the cover member.

The opening or openings 16, 18 in the cover member 12 can also take on a variety of different embodiments. According to one embodiment, the second opening can be sufficiently narrow to prevent the stethoscope head 20 from passing therethrough, yet may also be sufficiently large to allow the cover member to slide up and down the cord 22. This can secure the head cover to the stethoscope and prevent the head cover from falling off of the stethoscope and onto a surface that could expose the head cover to further contaminants.

In another embodiment, the second opening 16 can have a size that is substantially equal to the size of the opening 18. Alternatively, the first opening can be larger in size than the second opening. By way of exemplary embodiment and not by way of limitation, the first opening can be about 1.5 times larger than the second opening.

The size of either one or both openings can be adjustable by way of an adjustment member (not shown), although this is not required. According to one example, the adjustment member can be a drawstring or elastic band. Any applicable adjustment member known in the art may be incorporated into one or both of the openings 16, 18 of the cover member 12. The adjustment member can serve to make the second opening 16 sufficiently narrow to slidably engage a cord 22 associated with the head 20 of the stethoscope, and can also enlarge the second opening 16 so that the head cover 10 can be easily removed from the stethoscope altogether. Additionally, the first opening 18 may be equipped with an optional fastener that may be used to close the opening. Acceptable fasteners include but are not limited to hook and loop fasteners, clasps, buttons, zippers and snaps. A flap can be configured to fold over an outside portion of the cover member and substantially close the opening of the cover member. This flap can be an extension of or integral with the cover member. The head cover can also include magnets configured to enable substantial closure of the opening.

In one embodiment, the cover 12 may have a closable slit (not shown) that extends either part way, or entirely between and connects the two openings 16, 18. In such case, the cover can be partially or fully opened to disengage it completely from the stethoscope, for cleaning, treatment, disposal, etc., by unfastening and opening the slit. Fasteners, such as hook and loop fasteners, clasps, buttons, etc., can be coupled to the cover member along at least a portion of the perimeter surrounding the slit to close the slit.

As will be recognized by those having ordinary skill in the art, the head cover 10 can be made of a soft and flexible material. Alternatively, the cover member 12 can be made of a rigid or semi-rigid material. The cover member may be made from a number of suitable materials as required in order to achieve a specific result or desired purpose. The cover member may also be made water proof, either by the nature of the material selected, or a treatment applied thereto, and can also be made otherwise resistant to elements in order to protect the stethoscope head enveloped within the head cover. As such, the head cover may be made of both durable (i.e. rigid or semi-rigid) and soft flexible materials. Examples of suitable soft or flexible materials include without limitation, fabrics, such as wool, cotton, polyester, linen, rayon, fleece, and nylon, as well as polymeric materials, such as rubber, flexible plastics, neoprene, and vinyl. Further, natural materials, such as leather, or other skins may be used. Examples of durable (i.e. rigid or semi-rigid) materials include without limitation, plastics and other polymeric materials. Additionally, the cover member and/or the portion of the cover member surrounding the openings can be made reversibly deformable by including an elastic material in the composition of the head cover.

Whatever material is chosen for the cover member 12, it can be configured to provide the advantage of insulating the stethoscope head to keep it from becoming cold to the touch. In addition, as will be recognized by one of ordinary skill in the art, most if not all of the above-recited materials can be suited for printing or screening in order to accommodate specific designs or logos thereon.

The stethoscope head cover 10 of the present invention can provide a biocidal environment within the cavity 14 of the cover member 12. The biocidal environment can result from a number of features incorporated into the cover member. For example, an antimicrobial lining contiguous with an inner surface of the cover member may provide the biocidal environment. In another embodiment, the biocidal environment can be provided by the material of the cover member itself. In either case, the material or lining may be inherently biocidal and thus acts as a biocidal agent. Alternatively, the material or lining may be treated with or combined with a separate composition that acts as a biocidal agent. In a further embodiment, the lining or material of the cover member can act a reservoir to receive and at least temporarily hold a separate and distinct biocidal agent or antimicrobial solution, which can sanitize the stethoscope head 20 upon contact therewith. Regardless of the specific embodiment, the stethoscope head can be sanitized by coming into contact with the cover member itself, the antimicrobial lining or the biocidal agents associated therewith.

The antimicrobial lining (not shown) can take on a variety of embodiments and forms. For example, the antimicrobial lining can be a composition laminated on and secured to the inner surface of the cover member 12. Alternatively, the antimicrobial lining can be a fabric liner that may or may not be fixedly attached to the cover member itself. The lining may also be a chemical composition applied to the inner surface of the cover member. The lining itself can comprise a biocidal agent or alternatively can be configured to receive and hold a biocidal agent. Either way, the lining can effectively provide a biocidal environment to the cavity 14 within the cover member.

In a detailed aspect, the antimicrobial lining or material can be treated with the biocidal agent prior to manufacture of the head cover 10. Alternatively, the biocidal agent may be applied directly to the head cover by the consumer after manufacture of the head cover. Biocidal agents that may be particularly useful in connection with this embodiment include but are not limited to alcohols, such as ethanol, isopropyl, and mixtures thereof. By way of exemplary embodiment, the cover member 12 can be removed from the stethoscope head 20, inverted, and a biocidal agent, such as a disinfectant solution containing ethanol or isopropyl, can be sprayed or rubbed onto an inner surface of the cover member. Alternatively, the cover member can be immersed in or rinsed with the biocidal agent. The lining or cover member can be configured to at least temporarily hold the biocidal agent; however, to maintain a biocidal environment within the cavity 14 of the cover member, it may be necessary to reapply the biocidal agent throughout the life of the head cover.

In another aspect, ultra violet light (“UV light”), can be used in connection with the head cover 10 to provide a biocidal environment with in the cavity 14 of the cover member 12. UV light is known for its ability to sterilize surfaces and instruments, and is also a well-known viricide and bactericide. By way of specific example, a UV light source or plurality of UV light sources can be coupled to the cover member. A battery can be used to supply power to the UV light source. The battery can be sufficiently small so that it can be carried with or coupled to the head cover without adding substantial bulk or weight to the head cover. A switch can further be incorporated to activate the UV light source. The lining of the cover member can comprise a metallic foil that can further the distribution of UV light within the cavity of the cover member.

In still yet another embodiment, the antimicrobial lining can include fibers, such as metallic fibers, woven into the lining or the cover member 12. The fibers may have inherent antimicrobial properties. Such fibers are known in the art and include metallic fibers of copper, silver, gold and mixtures and composites thereof. In one example, silver in the form of silver ions, is integrated into a polymer from which the fibers are made. Alternatively, a metal, such as silver, can physically coat the fibers to make them antimicrobial.

Metals and/or their ions can be incorporated in a variety fashions other than as fibers as described above, to provide a biocidal environment to the head cover 10. For example, the head cover can be rinsed with or immersed in a solution containing silver ions. This can provide several days of antimicrobial protection to the head cover. The head cover can be removed from the stethoscope head 20 and periodically re-rinsed with the silver ion solution to renew its antimicrobial properties. In a further embodiment, a colloidal solution of a silver salt can applied directly to the lining adjacent the cavity of the cover member to provide a biocidal environment.

While a variety of metals, such as copper, gold and others not mentioned herein, can be used in connection with the head cover 10 of the present invention, metallic silver, silver oxides, silver salts and slightly soluble silver salts can be particularly useful in the present invention given their currently understood antimicrobial properties and methods of application.

Metals, including mixtures, composites and alloys thereof, which have antimicrobial properties, can be used substantially alone or in connection with other compositions to form biocidal agents suitable for use in connection with the head cover of the present invention. For example, silver can be used in connection with biopolymers, such as alginate, to form a biocidal agent, which can be incorporated into the cover member or antimicrobial lining.

Other biocidal agents, besides or in addition to metals and ions of metals, may be incorporated into the cover member 12 to provide a biocidal agent. Specifically, the biocidal agent can comprise at least one member selected from the group consisting of alcohols, quaternary ammonium salts, halides, biocidal polymers and mixtures thereof. Examples of halogens that may be used in providing the biocidal environment, include but are not limited to chlorine, bromine, iodine, fluorine and mixtures and composites thereof. Chlorine and bromine are particularly well-known for their ability to kill bacteria and act as a disinfectant.

By way of exemplary embodiment, chemical compositions such as heterocyclic and acyclic silane monomers and siloxane polymers, and their halogenated derivates can be used in connection with the fabric of the cover member 12 or lining to provide a biocidal environment within the cavity 14 of the cover member. These chemical compositions can be specifically of the class of biocidal monomers and polymers known as N-halamines. Examples of materials to which these chemical compositions can be applied include but are not limited to cellulose, chitin, synthetic fibers, plastics, rubber, vinyl, cellulosic fibers and blends containing cellulosic fibers.

In use, such chemical compositions can stabilize a biocidal agent, such as chlorine bleach, thereby locking the chlorine on the surface of the material. Chlorine and compositions containing chlorine are known to be effective biocidal agents. Unfortunately, chlorine can evaporate rather quickly. By using a material treated with N-halamine, the chlorine can be chemically bound to the fabric, thereby preventing it from evaporating or at least prolonging the useful biocidal life of the chlorine. In the event that the biocidal qualities of the head cover diminish, the head cover can be washed in a chlorine wash cycle to regenerate the biocidal effects thereof.

As set forth above, there are a number of embodiments by which the head cover 10 can provide a biocidal environment to the stethoscope head. Preferably, none of the embodiments, compositions or applications will adversely affect the integrity or longevity of the material comprising the cover member 12.

In addition to providing a biocidal environment within a cavity 14 of the cover member 12, the lining or cover member may comprise an insulating material, thereby keeping the stethoscope head 20 sufficiently warm to avoid an uncomfortable and cold sensation upon contact with a person's skin.

The present invention also provides a method of sanitizing a stethoscope head. The method can include providing a stethoscope head cover 12 in accordance with any of the embodiments set forth above, and engaging a stethoscope head 20 with the stethoscope head cover.

There has thus been outlined, rather broadly, various features of the invention so that the detailed description thereof that follows may be better understood, and so that the present contribution to the art may be better appreciated. Other features of the present invention will become clearer from the following detailed description of the invention, taken with the accompanying claims, or may be learned by the practice of the invention.

What is claimed is:

1. A stethoscope head cover configured to envelop a head of a stethoscope comprising:
(a) a cover member with a cavity therein, and an opening having a size sufficient to allow the head of the stethoscope to be passed therethrough; and
(b) a biocidal environment within the cavity provided by an antimicrobial lining contiguous with an inner surface of the cover member.

2. The stethoscope head cover of claim 1, wherein the antimicrobial lining is an insulating material.

3. The stethoscope head cover of claim 1 wherein the antimicrobial lining comprises a biocidal agent.

4. The stethoscope head cover of claim 3, wherein the antimicrobial lining is configured to at least temporarily hold the biocidal agent.

5. The stethoscope head cover of claim 3 wherein the biocidal agent comprises at least one member selected from the group consisting of: alcohols, quaternary ammonium salts, metals, halides, biocidal polymers, and mixtures thereof.

6. The stethoscope head cover of claim 3, wherein the biocidal agent comprises a metal.

7. The stethoscope head cover of claim 6, wherein the metal is a member selected from the group consisting of: copper, silver, gold and mixtures, composites, and alloys thereof.

8. The stethoscope head cover of claim 3, wherein the biocidal agent comprises biocidal polymers.

9. The stethoscope head cover of claim 3, wherein the biocidal agent comprises an alcohol.

10. The stethoscope head cover of claim 9, wherein the alcohol is a member selected from the group consisting of: ethanol and isopropyl, and mixtures thereof.

11. The stethoscope head cover of claim 3 wherein the biocidal agent comprises at least one member selected from the group consisting of chlorine and bromine and combinations and mixtures thereof.

12. The stethoscope head cover of claim 1 wherein the cover member further comprises a second opening with a size sufficient to slidably engage a cord associated with the head of the stethoscope.

13. The stethoscope head cover of claim 12, wherein the second opening is sufficiently narrow to prevent the stethoscope head from passing therethrough.

14. The stethoscope head cover of claim 12, wherein the second opening has a size that is substantially equal to the size of the opening.

15. The stethoscope head cover of claim 12, wherein the size of the opening is about 1.5 times larger than the size of the second opening.

16. The stethoscope head cover of claim 1, wherein the opening can be fastened into a substantially closed position.

17. The stethoscope head cover of claim 1, wherein the cover member comprises a soft and flexible material.

18. The stethoscope head cover of claim 1, wherein the soft and flexible material includes a polymeric material.

19. The stethoscope head cover of claim 17, wherein the soft and flexible material includes a fabric material.

20. A stethoscope head cover configured to envelop a head of a stethoscope comprising:
(a) a cover member with a cavity therein, and an opening having a size sufficient to allow the head of the stethoscope to be passed therethrough; and
(b) a biocidal environment within the cavity of the cover member.

21. The stethoscope head cover of claim 20, wherein the biocidal environment is provided by a material of the cover member.

22. A stethoscope head cover configured to envelop a head of a stethoscope comprising:
(a) a cover member with a cavity therein, said cover member further having;
(b) a first opening having a size sufficient to allow the head of the stethoscope to be extended therethrough; and
(c) a second opening with a size sufficient to slidably engage a cord associated with the head of the stethoscope.

23. The stethoscope head cover of claim 1, wherein the second opening is sufficiently narrow to prevent the stethoscope head from passing therethrough.

24. The stethoscope head cover of claim 1, wherein the size of the opening is about 1.5 times larger than the size of the second opening.

25. A method of sanitizing a stethoscope head comprising:
(a) providing a stethoscope head cover, the head cover comprising:
(i) a cover member with a cavity therein, and an opening having a size sufficient to allow the head of the stethoscope to be passed therethrough; and
(ii) a biocidal environment within the cavity of the cover member; and
(b) engaging a stethoscope head with the stethoscope head cover.
